(19) United States
(12) Patent Application Publication
(10) Pub. No.: US 2025/0155370 A1
(43) Pub. Date: May 15, 2025

(54) Method for Determining the Quantitative Ratio of Melamine-Formaldehyde Resin and Urea-Formaldehyde Resin in at Least One Paper Layer Impregnated With a Mixture of These Resins

(71) Applicant: Flooring Technologies Ltd., Kalkara (MT)

(72) Inventors: Jinming Zhang, Berlin (DE); Norbert Kalwa, Horn-Bad Meinberg (DE)

(21) Appl. No.: 18/287,506

(22) PCT Filed: Apr. 20, 2022
(86) PCT No.: PCT/EP2022/060334

§ 371 Date: Oct. 11, 2024

(30) Foreign Application Priority Data
Apr. 22, 2021 (EP) 21169864.2

(51) Int. Cl. G01N 21/359; G01N 21/3563

(52) U.S. Cl. CPC G01N 21/359; G01N 21/3563

(57) ABSTRACT

The present invention relates to a method for determining the quantitative ratio of melamine-formaldehyde resin and urea-formaldehyde resin in at least one paper layer impregnated with a mixture of these resins, wherein the impregnation is carried out in a plant for impregnating at least one paper layer passing through the plant, comprising the steps Impregnating of several paper layers with resin mixtures, each with different quantitatively defined ratios of melamine-formaldehyde resin and urea-formaldehyde resin as reference samples; Recording of at least one NIR spectrum of each of the reference samples using at least one NIR measuring head in a wavelength range between 500 nm and 2500 nm, preferably between 700 nm and 2000 nm, particularly preferably between 900 nm and 1700 nm, and especially advantageously between 1450 nm and 1550 nm; Correlating the different quantitative ratios of melamine-formaldehyde resin and urea-formaldehyde resin in the reference samples to the recorded NIR spectra of said reference samples; and Establishing a calibration model for the correlation between the spectral data of the NIR spectra and the associated quantitative ratios of melamine-formaldehyde resin and urea-formaldehyde resin of the reference samples by means of multivariate data analysis; Impregnating at least one paper layer with a resin mixture of melamine-formaldehyde resin and urea-formaldehyde resin; Recording at least one NIR spectrum of the impregnated paper layer using the at least one NIR measuring head in a wavelength range between 500 nm and 2500 nm, preferably between 700 nm and 2000 nm, in particular preferably between 900 nm and 1700 nm and especially advantageously between 1450 nm and 1550 nm; and Determining the quantitative ratio of melamine-formaldehyde resin and urea-formaldehyde resin in the impregnated paper layer by comparing the NIR spectrum recorded for the impregnated paper layer with the calibration model created.

The present invention relates to a method for determining the quantitative ratio of melamine-formaldehyde resin and urea-formaldehyde resin in at least one paper layer impregnated with a mixture of these resins and an impregnation plant for carrying out said method and the use of the data determined for controlling such impregnation plant.

### DESCRIPTION

In the wood-based materials industry, impregnated papers are used on a large scale for the surface finishing of wood-based panels. These are mainly decorative papers, overlays and counteracting papers. These impregnated papers are pressed onto wood-based materials in so-called short-cycle presses (KT presses) at high pressure and high temperatures. These coated boards are then used in a wide variety of applications (furniture, interior finishing, laminate flooring, etc.).

Water-based melamine and urea resins are mainly used to impregnate the papers. The cheaper urea resins are mostly used in the so-called core impregnation of the papers, mixed with melamine resins, whereas pure melamine resins are mostly used on the surface of the papers.

In the production of impregnates, the urea resin with a melamine content is applied in a first impregnation tank where, after immersion in the impregnation tank, the resin is wiped off to a defined resin coating by means of doctor blades or squeegee rollers. The impregnate is then dried with hot air in a floating dryer and then a top coat of melamine resin is applied followed by further drying. By way of example, reference is made to EP 3075906 B1, which describes an impregnation plant consisting of a first impregnation station with a downstream drying station and a second impregnation station with a downstream drying station.

In the impregnation process, it is difficult to determine the ratio of the application quantities of the resins after the first tank. The determination can only be made by taking a sample from the running process, whereby there is a risk of paper tears due to the high speed of the plant (60-100 m/min) and the fact that the paper is moist. In addition, the sample can only be taken from the edge areas of the web.

Another possibility would be to first carry out only a pre-/core impregnation and to first check the parameters of the pre-impregnation there. However, this leads to considerable material losses, as these impregnates cannot be used. In addition, this process would have to be repeated every time a change is made to very different papers.

In addition, only a statement about the total amount of resin can be made. Although the proportions should be determined by given formulations, they can deviate from the specifications for a variety of reasons (incorrect formulations, dosing and mixing problems).

Of course, when impregnating papers, variations in the raw materials (e.g. resins with different solid contents), production parameters or production errors can also result in products with unclear quantity ratios in the core of the impregnates. It can lead to the fact that the product quality could be different from batch to batch. Of course, this also has an influence on the costs and the quality of the product, which can be critical for the mass product impregnate.

The resulting disadvantages are more difficult error analysis, rising costs and difficult quality control.

The invention is therefore based on the technical task of developing a method that allows the ratio between urea and melamine resin to be quickly tested quantitatively in the impregnated papers. The method should serve to monitor production. The test should be non-destructive, should be possible over the entire web width and should provide continuous data.

This task is solved by a method with the features of claim 1.

Accordingly, there is provided a method for determining the quantitative ratio of melamine and urea-formaldehyde resin in at least one paper layer impregnated with a mixture of said resins, wherein said impregnation is carried out in a plant for impregnating at least one paper layer passing through said plant. The present process comprises the following steps:
Impregnating of several paper layers with resin mixtures, each with different quantitatively defined ratios of melamine-formaldehyde resin and urea-formaldehyde resin as reference samples;
Recording of at least one NIR spectrum of each of the reference samples using at least one NIR measuring head in a wavelength range between 500 nm and 2500 nm, preferably between 700 nm and 2000 nm, particularly preferably between 900 nm and 1700 nm, and especially advantageously between 1450 nm and 1550 nm;
Correlating the different quantitative ratios of melamine-formaldehyde resin and urea-formaldehyde resin in the reference samples to the recorded NIR spectra of said reference samples; and
Establishing of a calibration model for the correlation between the spectral data of the NIR spectra and the associated quantitative ratios of melamine and urea-formaldehyde resin of the reference samples by means of multivariate data analysis;
Impregnating of at least one paper layer with a resin mixture of melamine and urea-formaldehyde resin;
Recording at least one NIR spectrum of the impregnated paper layer using the at least one NIR measuring head in a wavelength range between 500 nm and 2500 nm, preferably between 700 nm and 2000 nm, in particular preferably between 900 nm and 1700 nm and especially advantageously between 1450 nm and 1550 nm; and
Determining the quantitative ratio of melamine and urea-formaldehyde resin in the impregnated paper layer by comparing the NIR spectrum recorded for the impregnated paper layer with the calibration model created.

Thus, a NIR measuring head is used which, by recording spectral data (spectra) in the near infrared range (950-1650 nm), allows the determination of melamine resin impregnated papers containing certain or uncertain amounts of the urea resin. The NIR radiation interacts with the organic functional groups, such as O—H, C—H and N—H, which are present in both urea and melamine resin. During the interaction, the NIR radiation is scattered and reflected by the measured sample. By receiving the reflected NIR radiation by an NIR detector, an NIR spectrum is generated. Dozens of NIR radiations are performed in one second during the measurement, so that a static validation of the values is also ensured. The present method enables quantitative detection of the urea resin in melamine resin impregnated papers.

Urea and melamine resins have very similar NIR spectra, with the urea resin peak lying in the shoulder of the melamine resin peak. Due to the superposition of the resins used, there is an attenuation of the urea resin peak in the spectrum, which makes the analysis more difficult. For this reason, it was surprising that it was possible to detect the urea resin in melamine resin impregnated papers at all.

Although the determination of the concentrations of different resin types, wax, moisture and other additives by NIR spectroscopy in resin-wood composites is known (see US 2007/0131862 A1), the determination of the concentrations of the mentioned components is carried out on a mixture of (isolated) wood particles and binder before pressing this mixture into corresponding wood composites or wood products. In contrast, the present method allows the determination of the quantitative ratio of melamine-formaldehyde resin and urea-formaldehyde resin in an impregnated paper layer after intermediate drying.

In one embodiment of the present method, the determination of the quantitative ratio of melamine-formaldehyde resin and urea-formaldehyde resin in an impregnated paper layer is carried out after a drying step, in particular an intermediate drying step, wherein the water content of the paper layer is preferably reduced to 10-20 wt %.

In a further embodiment of the present method, the quantitative ratio of melamine and urea-formaldehyde resin is determined in at least one paper layer pre-impregnated (or core-impregnated) with this resin mixture.

In a still further preferred embodiment of the present method, the determination of the quantitative ratio of melamine and urea-formaldehyde resin in at least one paper layer pre-impregnated (or core-impregnated) with this resin mixture is carried out after leaving a (first) impregnation station as part of a plant for impregnating at least one paper layer passing through the plant. Preferably, after pre-impregnation of the paper layer in a first impregnation station, surface impregnation of the pre-impregnated paper layer takes place in a second impregnation station. Such an impregnation plant is described in detail below.

The determination of the quantitative ratio of melamine-formaldehyde resin and urea-formaldehyde resin is thus carried out between pre-impregnation and surface impregnation of the paper web running through the impregnation plant, i.e. between the first and second impregnation stations.

The present method requires only a single NIR measurement to determine the quantitative ratio of melamine-formaldehyde resin and urea-formaldehyde resin. It is also advantageous that the measurement is carried out on a continuous paper layer, i.e. the paper layer is not destroyed.

According to the present method, reference samples of the paper layer impregnated with a defined mixture of melamine and urea-formaldehyde resin are first provided. It is essential that the reference sample is similar to the sample to be measured; i.e. in particular the resin mixture of the reference sample has the same composition as the resin mixture to be measured. The similarity of the sample to be measured and the reference sample is particularly important when using resin mixtures with additives such as flame retardants, fibres and other additives.

At least one NIR spectrum is recorded from these reference samples in a wavelength range between 500 nm and 2500 nm, preferably between 700 nm and 2000 nm, in particular preferably between 900 nm and 1700 nm.

The quantitative composition of the resin mixtures used for the reference samples is known. Thus, the quantitative ratio of melamine-formaldehyde resin and urea-formaldehyde resin in the resin mixture used for pre-impregnation may be between 90% by weight:10% by weight and 10% by weight:90% by weight, preferably between 75% by weight:25% by weight and 25% by weight:75% by weight, more preferably between 55% by weight:45% by weight and 45% by weight:55% by weight.

Also, the resin content in an impregnated paper layer is >100%. If the paper weight is taken as a basis, the resin content is usually around 100-140%, as is the case with decorative impregnates. With more special impregnates, the resin content can be significantly higher, such as with overlay paper impregnates.

The known quantitative composition of the resin mixture is then assigned to the respective NIR spectra of these reference samples, and a calibration model is created for the correlation between the spectral data of the NIR spectra of the reference samples and the associated parameter values by means of multivariate data analysis; i.e. an NIR spectrum of the reference sample corresponds to each parameter value of the reference sample. The calibration models created for the different parameters are stored in a suitable data memory.

Subsequently, at least one paper layer is (pre-) impregnated with a resin mixture of melamine and urea-formaldehyde resin and at least one NIR spectrum of the (pre-) impregnated paper layer is recorded. The quantitative ratio of melamine and urea-formaldehyde resin in the (pre-) impregnated paper layer can then be determined by comparing the NIR spectrum recorded for the resin layer with the calibration model created.

It makes sense to compare and interpret the NIR spectra over the entire recorded spectral range. This is advantageously carried out with a multivariate data analysis (MDA) known per se. In multivariate analysis methods, several statistical variables are typically examined simultaneously in a manner known per se. For this purpose, the number of variables contained in a data set is usually reduced without simultaneously reducing the information contained therein.

In the present case, the multivariate data analysis is carried out using the partial least squares regression (PLS) method, which allows a suitable calibration model to be created. The evaluation of the data obtained is preferably carried out with suitable analysis software, such as the analysis software SIMCA®-P from Umetrics AB or The Unscrambler® from CAMO.

In another embodiment, it is intended to use spectral data from the NIR spectral range between 1450 and 1550 nm for the creation of the calibration model, which are pre-treated by means of suitable mathematical methods and then fed to the multivariate data analysis.

The significance of a wavelength for the prediction of the resin ratio from the NIR spectrum is shown with the help of the regression coefficients. The regions with large coefficient amounts have a strong influence on the regression model. For example, the representation of the regression coefficients in a PLS regression model for the determination of the resin quantity or resin content shows that the wavelength range between 1460 nm and 1530 with a maximum at 1490 nm (absorption band of the amino groups of the resin) is the most important for the calculation of the model, as the amounts of the regression coefficients are largest here. The other ranges in the spectrum have less information content in relation to the NIR measurement, but nevertheless contribute to taking into account or minimizing the other information or interfering influencing variables (such as transparency of the layer, surface properties of the substrate material, etc.).

To eliminate interfering influences (such as the nature of the surface of the substrate, the colour of the samples, light scattering from solid particles or other additives, etc.), it is necessary to process the spectral data using mathematical pre-treatment methods (e.g, derivative data pre-treatment, standardization according to SNVT (Standard Normal Variate Transformation), multiplicative signal correction (EMSC, Extended Multiplicative Signal Correction, etc.). In this process, the baseline effects, which are mainly caused by the different colour of the samples, are removed from the spectra, overlapping bands are separated from each other and the dependence of the light scattering on the substrate surface or on the solid particles in the coating is taken into account. When measuring a decorative paper layer, the focus of calibration and data pre-treatment is on removing the baseline shift.

From the pre-treated data, a calibration model is developed using multivariate data analysis, which includes all decors used in the calibration.

Accordingly, the comparison and interpretation of the NIR spectra are preferably carried out in the spectral range between 1450 and 1550 nm using multivariate data analysis MDA. In multivariate analysis methods, several statistical variables are typically examined simultaneously in a manner known per se. For this purpose, the number of variables contained in a data set is reduced without simultaneously reducing the information contained therein.

As described above, the ratio of melamine-formaldehyde resin and urea-formaldehyde resin in a (pre-) impregnated paper layer is determined. The paper layers used are, for example, overlay papers, decorative papers or kraft papers. Overlay papers are thin papers that have typically already been impregnated with a conventional melamine resin. Overlay papers are also available in which abrasion-resistant particles, such as corundum particles, are mixed into the resin of the overlay or are sprinkled onto the impregnated overlay to increase abrasion resistance. Decor papers are special papers for surface finishing of wood-based materials, which allow a high variety of decors. In addition to the typical imprints of various wood structures, more extensive imprints of geometric shapes or artistic, decorative designs are available. In fact, there is no restriction in the choice of motifs. To ensure optimal printability, the paper used must have good smoothness and dimensional stability and also be suitable for penetration of a necessary synthetic resin impregnation. Kraft papers have a high strength and consist of cellulose fibres to which starch, alum and glue have been added to increase strength.

In a preferred embodiment, the paper layer is partially or completely impregnated with the resin, whereby the resin penetrates into the substrate. As used herein, the term “impregnation” is to be understood as a complete or partial impregnation of the paper layer with the resin. In particular, the present method is used for pre-impregnated or core-impregnated paper layers.

The present method for determining the quantitative ratio of melamine and urea-formaldehyde resin in a pre-impregnated paper layer can be carried out continuously and online in an impregnation plant.

A corresponding impregnation plant for impregnating at least one paper layer passing through the system comprises:
optional paper unwind and changing device,
a first impregnation station for pre-impregnation (core impregnation) of at least one paper layer with a resin mixture of melamine-formaldehyde resin and urea-formaldehyde resin, and
a second impregnation station for surface impregnation of the at least one pre-impregnated paper layer,
wherein at least one NIR measuring head, in particular at least one NIR multi measuring head, is provided between the first impregnation station and the second impregnation station for recording at least one NIR spectrum of the impregnated paper layer after the pre-impregnation in the first impregnation station,
optional clipper and deposit table

The NIR measuring head is preferably arranged in such a way that the upper side of the impregnated paper layer is irradiated. In general, however, it is also possible to provide additional NIR measuring heads in the impregnation plant for measuring the top and bottom of the impregnated paper layer.

It is particularly preferred if the at least one NIR measuring head traverses across the width of the paper layer and analyses specific problem areas (e.g. in the edge or middle area of the boards, etc.). Accordingly, the at least one NIR measuring head moves transversely to the running direction of the (pre-) impregnated paper layer. In addition, the measured values are immediately available and allow immediate intervention in the process. This is not readily possible with other methods.

In an embodiment of the impregnation plant, a first drying station is provided downstream of the first impregnation station, wherein the at least one NIR measuring head is arranged downstream of the first drying station.

In a specific embodiment, the impregnation plant comprises impregnation tanks or impregnation dipping baths (as impregnation stations), optionally a breathing section, a doctor system/squeezing roller pair for removing excess resin, optionally a device for scattering abrasion-resistant particles, at least one dryer (e.g. a floating dryer), optionally a grid unit and optionally a second dryer, at least one cooling device (e.g. a cooling roller system).

A method is thus provided in which, by using an NIR measuring head, the quantitative ratio of melamine-formaldehyde resin and urea-formaldehyde resin in a (pre-) impregnated paper layer can be determined from an NIR spectrum, by means of a non-contact measurement. The data determined with the measuring head or heads can be used directly for plant control or regulation.

In addition, in further preferred embodiment the storage of data can enable an improvement in quality control. The stored data can also advantageously contribute to the evaluation of plant tests, e.g. commissioning of a plant during new installation or after maintenance or repair or for in-situ testing purposes of new production or measuring processes. The immediate availability of the measured values and the high measuring frequency enable very close monitoring or control or regulation of the systems.

The present method enables the provision of the measured values in a short time (online, preferably without disturbing time delay) compared to conventional (known) measuring methods. The measurement data can be used for quality assurance, research and development, process control, process regulation, process control, etc. The measurement process does not reduce the production speed, etc. Basically, it improves the monitoring of production. In addition, downtimes due to quality determinations and plant adjustments are also reduced.

The advantages of the present method are manifold: non-contact multi-parameter determination (“on-time” or “real-time” measurement) with significantly reduced time delay in the evaluation of the measured parameter values; improved plant control or regulation, reduction of rejects, improvement of the quality of the products manufactured on the plant, improvement of the plant availability.

The control system of the impregnation plant comprises at least one computer-aided evaluation unit (or processor unit) and a database. In the evaluation unit, the NIR spectrum measured for the product (i.e. pressed porous coating material) is compared with the calibration models created for the individual parameters. The parameter data determined in this way are stored in the database.

The data determined with the present spectroscopic method can be used to control impregnation plants. The non-contact measured parameter values of the NIR multi-measurement head (“actual values”) can be used directly and in “real time” for the control or regulation of the plant, as already described, for example by storing the measured actual values stored in the database, e.g. a relational database, and comparing them with target values of these parameters existing there. The resulting differences are then used to control or regulate the production line.

A computer-implemented method and a computer program comprising instructions which, when executed by a computer, cause the computer to execute the computer-implemented method, may be provided for balancing and controlling the impregnation plant. The computer program is stored in a memory unit of the impregnation equipment control system.

The invention is explained in more detail below using examples of embodiments.

### Production of the Impregnated Papers

To produce the impregnated papers, the decorative paper (paper weight:65 g/m², 30×30 cm) is impregnated with the following 3 mixing ratios of urea resin (HF, Metadynea Primere 70 1093L, solid resin content: 52 wt %) and melamine resin (MF, Metadynea Primere 700867L, solid resin content: 62 wt %) (see Table 1).

TABLE 1
3 Mixing ratios for resin mixing of MF(wt %) and HF(wt %)

| | Resin | | | | | |
|---|---|---|---|---|---|---|
| | mixture | | MF in | | | |
| | MF | MF | resin | HF | HF | |
| | and HF | (in | (in g | (in | (in g | MF (wt %)/ |
| Variant | (in g) | g fl. ) | solid) | g fl. ) | solid) | HF (wt %) |
| 1 | 10 | 10 | 6.2 | 0 | 0 | 100*:0 |
| 2 | 10 | 8 | 4.96 | 2 | 1.04 | 83*:17 |
| 3 | 10 | 2 | 1.24 | 8 | 4.16 | 25*:75 |

*MF wt % is used as a reference value to create the calibration model

For each impregnation, 10 g of resin mixture is applied to the decor paper. To accelerate the curing of the resin, 0.1 g hardener (Alton HM 1448) is added to the resin mixture. The impregnated papers are dried in the drying cabinet to approx. 6 wt %.

### Creation of the Calibration Model

The impregnated paper as reference sample is measured by NIR. In order to create a good statistical calibration model, several measuring points (approx. 15) are measured on the sample when recording the NIR spectra. For each measuring point, 2 measurements are repeated. The NIR spectra were recorded in the laboratory with the DA 7250 measuring system (Perten Instruments GmbH).

After recording the NIR spectra, MF is correlated with spectral data in wt %. The calibration model is created using multivariate data analysis. This is done with suitable analysis software, for example “The Unscrambler®” from CAMO.

A calibration model is created from the reference spectra, which can be used to determine (predict) the quantitative testing of the urea resin of an unknown sample.

A total of 15 NIR spectra are used to create the calibration model. 7 NIR spectra of variant 1 are recorded. 4 NIR spectra each are recorded from variant 2 and variant 3. All 15 NIR spectra are performed with data pre-treatments Detrend. The PLS model is then created. The parameters of the calculated model are summarised in Table 2.

TABLE 2
PLS model for the quantitative determination of urea resin in
melamine resin impregnated papers

| | Data | | | | | | |
|---|---|---|---|---|---|---|---|
| Model | pre-treatment | PLS | RMSECV | R² | Bias | Slope | Offset |
| 1 | Detrend | 3* | 3.94 | 0.98 | −0.33 | 0.98 | 0.59 |

*Optimum number of main factors suggested by The Unscrambler programme

### Validation of the Calibration Model

To test the above calibration model, the impregnate of variants 2 and 3 described in Table 1 is prepared again with the above resin ratios. In addition, a 10 g resin mixture of 5 g MF and 5 g HF is prepared. This new mixture is referred to as ratio 4. The ratio 4 serves as an unknown sample for testing the calibration model. The following parameters of the unknown sample can be taken from Table 3.

TABLE 3
Mixing ratio 4 for validation of the calibration model

| | MF | | Solid resin | | Solid resin | |
|---|---|---|---|---|---|---|
| | and | | content of MF | | content of HF in | MF |
| Mixing | HF( in | Weight of | in the resin | Weight of | the resin mixture | (wt %)/ |
| ratio | g fl. ) | MF(g) | mixture (g) | HF(g) | (g) | HF (wt %) |
| 4 | 10 | 5 | 3.1 | 5 | 2.6 | 54*:46 |

*MF in wt % is used as reference value for testing the calibration model

With the mixing ratio 2, 3 and 4, 2 samples each are produced by means of the production processes described above in order to test the calibration model. From each sample, a measured value of the MF wt % is determined by means of the calibration model. In total, 6 measured values are obtained from 6 samples (see Table 4). The MF mean value of both sample measurements is determined.

TABLE 4
Summary of the validation of the first calibration model.

| | | | | | Deviation |
|---|---|---|---|---|---|
| | | Measured | Measured | | between |
| | | value of | value of | | reference |
| | Reference | MF of | MF of | MF average | value and |
| | value of | sample 1 | sample 2 | of samples 1 | mean value |
| Variant | MF(wt %) | (wt %) | (wt %) | and 2 (wt %) | (wt %) |
| 2 | 83 | 70 | 72 | 71 | −12 |
| 3 | 25 | 14 | 16 | 15 | −10 |
| 4 | 54 | 48 | 50 | 49 | −5 |

Although the deviation between the reference value and the mean value of the measurements of mixing ratio 2 and 3 is greater than 10% by weight, the deviation between the reference value and the mean value of the measurements of mixing ratio 4 is 5% by weight significantly smaller than that of mixing ratio 2 and 3.

From practical experience, a resin mixture between MF and HF of up to 50%: 50% is used in impregnated paper during pre-impregnation. These results from Table 4 show that when the resin mix ratio of MF and HF is 50 wt %, the calibration model can accurately determine the quantitative amounts of urea in melamine impregnated paper.

The on-line measurement of the resin ratios takes place after the first dryer, as the amount of water is reduced to approx. 10 to 20 wt % there and no longer overlays the urea peak. The measuring head should traverse the paper web to also detect differences across the width.

1. A method for determining the quantitative ratio of melamine-formaldehyde resin and urea-formaldehyde resin in at least one paper layer impregnated with a mixture of these resins, wherein the impregnation is carried out in a plant for impregnating at least one paper layer passing through the plant,
comprising the steps of:
impregnating of several paper layers with resin mixtures, each with different quantitatively defined ratios of melamine-formaldehyde resin and urea-formaldehyde resin as reference samples;
recording of at least one NIR spectrum of each of the reference samples using at least one NIR measuring head in a wavelength range between 500 nm and 2500 nm, preferably between 700 nm and 2000 nm, particularly preferably between 900 nm and 1700 nm, and especially advantageously between 1450 nm and 1550 nm;
correlating the different quantitative ratios of melamine-formaldehyde resin and urea-formaldehyde resin in the reference samples to the recorded NIR spectra of said reference samples;
establishing a calibration model for the correlation between the spectral data of the NIR spectra and the associated quantitative ratios of melamine-formaldehyde resin and urea-formaldehyde resin of the reference samples by means of multivariate data analysis;
impregnating at least one paper layer with a resin mixture of melamine-formaldehyde resin and urea-formaldehyde resin;
recording at least one NIR spectrum of the impregnated paper layer using the at least one NIR measuring head in a wavelength range between 500 nm and 2500 nm, preferably between 700 nm and 2000 nm, in particular preferably between 900 nm and 1700 nm and especially advantageously between 1450 nm and 1550 nm; and
determining the quantitative ratio of melamine-formaldehyde resin and urea-formaldehyde resin in the impregnated paper layer by comparing the NIR spectrum recorded for the impregnated paper layer with the calibration model created.

2. The method according to claim 1, wherein the determination of the quantitative ratio of melamine-formaldehyde resin and urea-formaldehyde resin is carried out in a pre-dried impregnated paper layer.

3. The method according to claim 1, wherein the determination of the quantitative ratio of melamine-formaldehyde resin and urea-formaldehyde resin is carried out in at least one paper layer pre-impregnated (or core-impregnated) with said resin mixture.

4. The method according to claim 1, wherein the determination of the quantitative ratio of melamine-formaldehyde resin and urea-formaldehyde resin in at least one paper layer pre-impregnated (or core-impregnated) with said resin mixture is performed after leaving a (first) impregnation station as part of a plant for impregnating at least one paper layer passing through said plant.

5. The method according to claim 4, wherein, following pre-impregnation of the paper layer in a first impregnation station, surface impregnation of the pre-impregnated paper layer takes place in a second impregnation station.

6. The method according to claim 1, wherein the determination of the quantitative ratio of melamine-formaldehyde resin and urea-formaldehyde resin in a pre-impregnated paper layer is carried out continuously and online.

7. The method according to claim 1, wherein the quantitative ratio of melamine-formaldehyde resin and urea-formaldehyde resin in the resin mixture used for pre-impregnation is between 90% by weight:10% by weight and 10% by weight:90% by weight, preferably between 75% by weight:25% by weight and 25% by weight:75% by weight, more preferably between 55% by weight:45% by weight and 45% by weight:55% by weight.

8. The method according to claim 1, wherein spectral data from the entire recorded spectral range are used to create the calibration model.

9. The method according to claim 1, wherein spectral data from the NIR spectral range between 1450 nm and 1550 nm are used for the creation of the calibration model, which are pre-treated by means of suitable mathematical methods and are subsequently fed to the multivariate data analysis.

10. An impregnation plant for impregnating at least one paper layer passing through the system comprises:
a first impregnation station for pre-impregnation (core impregnation) of at least one paper layer with a resin mixture of melamine-formaldehyde resin and urea-formaldehyde resin, and
a second impregnation station for surface impregnation of the at least one pre-impregnated paper layer,
wherein at least one NIR measuring head, in particular at least one NIR multi measuring head, is provided between the first impregnation station and the second impregnation station for recording at least one NIR spectrum of the impregnated paper layer after the pre-impregnation in the first impregnation station.

11. The impregnation plant according to claim 10, wherein, a first drying station is provided downstream of the first impregnation station, wherein the at least one NIR measuring head is arranged downstream of the first drying station.

12. The impregnation plant according to claim 10, wherein the at least one NIR measuring head moves transversely over the width of the paper and analyses specific problem areas.

13. The method according to claim 1 for controlling impregnation plants by using the determined data.
